(19) United States
(12) Patent Application Publication
(10) Pub. No.: US 2006/0173074 A1
(43) Pub. Date: Aug. 3, 2006

(54) Treatment of restless legs syndrome

(76) Inventors: Juha Ellmen, Turku (FI); Johanna Karvinen, Turku (FI); Mikko Vahteristo, Kuopio (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/269,547

(22) Filed: Nov. 9, 2005

Related U.S. Application Data
(60) Provisional application No. 60/626,447, filed on Nov. 10, 2004.

(51) Int. Cl. A61K 31/277; A61K 31/198; A61K 31/137

(52) U.S. Cl. 514/521; 514/649; 514/567

(57) ABSTRACT

A method for the treatment of restless legs syndrome in a patient using a combination of a COMT-inhibitor, a decarboxylase inhibitor and a dopamine precursor.

This application claims the benefit of priority to U.S. provisional application No. 60/626,447, filed on Nov. 10, 2004, the contents of which are incorporated by reference herein.

### FIELD OF THE INVENTION

The present invention relates to a method for the treatment of restless legs syndrome (RLS). More particularly, the present invention relates to the treatment of restless legs syndrome by using the combination of a catechol-O-methyl transferase (COMT) inhibitor, a peripheral decarboxylase (DDC) inhibitor and a dopamine precursor.

### BACKGROUND OF THE INVENTION

RLS, also known as Ekbom's syndrome, is a neurological disorder and has an estimated prevalence of 2.5 to 15% in the adult population; the prevalence increases with age and is higher in women than in men. The condition is characterized by an irresistible urge to move the legs (akathisia), accompanied by other unpleasant sensations deep within the legs. The symptoms, which may extend to involve the arms or the trunk, are relieved by movement. The circadian rhythm seems to influence the subjective sensory symptoms of RLS and the associated periodic limb movements (PLM), which occur in approximately 80% of RLS patients. They occur primarily in the evening and at night, and the likelihood of symptoms increases with the relaxation of the patient.

The idiopathic or primary form of RLS occurs without any indication of other disease processes. Secondary or symptomatic RLS is known to occur in relation to a number of other medical conditions. The conditions that have been found to be clearly associated with symptomatic RLS include renal failure, as in end-stage renal disease, iron deficiency with or without anemia, pregnancy, and neuropathy or radiculopathy. Other associations of possible clinical importance include rheumatoid arthritis, diabetes, and both folate and magnesium deficiencies.

There is no officially established treatment of choice for RLS. For those who do receive RLS treatment, the medicinal products are usually prescribed “off-label”. Dopaminergic products, especially dopamine agonists and dopamine precursors, including levodopa, appear to be the most common of the drug treatments currently used in RLS (Brodeur C, Montplaisir J, Godbout R, Marinier R, Neurology 1988:38:1845-1848, Kaplan P W, Allen R P, Buchholz D W, Walters J K, Sleep 1993;16:717-723, Trenkwalder C, Stiasny K, Pollmacher T et al., Sleep 1995;18(8):681-688, Benes H, Kurella B, Kummer J, Kazenwadel J, Selzer R, Kohnen R, Sleep 1999;22(8):1073-1081). A limitation of levodopa/DDC inhibitor treatment in RLS is that it is relatively short-acting, due to the short half-life of levodopa in serum, and therefore it provides less symptomatic relief during the second half of the night (Benes H et al. ibid). Ashafq A, Sharif M D, Movement Disorders 2003;17(2):421 discloses the use of a COMT inhibitor in combination with levodopa in the treatment of secondary RLS after renal failure in a patient with polycystic kidney disease where alleviaton of symptoms was reported up to 5 hours with a combination of a COMT inhibitor and up to 200 mg of sustained release levodopa/carbidopa.

### BRIEF DESCRIPTION OF THE DRAWINGS

FIG. 1 shows the PLM-indexes (periodic limb movement indexes) during the first (1^{st}) and second (2^{nd}) half of a polysomnography night in 28 patients with RLS undergoing a 7 period cross-over study.

FIG. 2 shows the hourly PLM-indexes during a polysomnography night in 28 patients with RLS undergoing a 5 period cross-over study. The abbreviations PSG and TIB mean polysomnography and total time in bed, respectively.

### DETAILED DESCRIPTION OF THE INVENTION

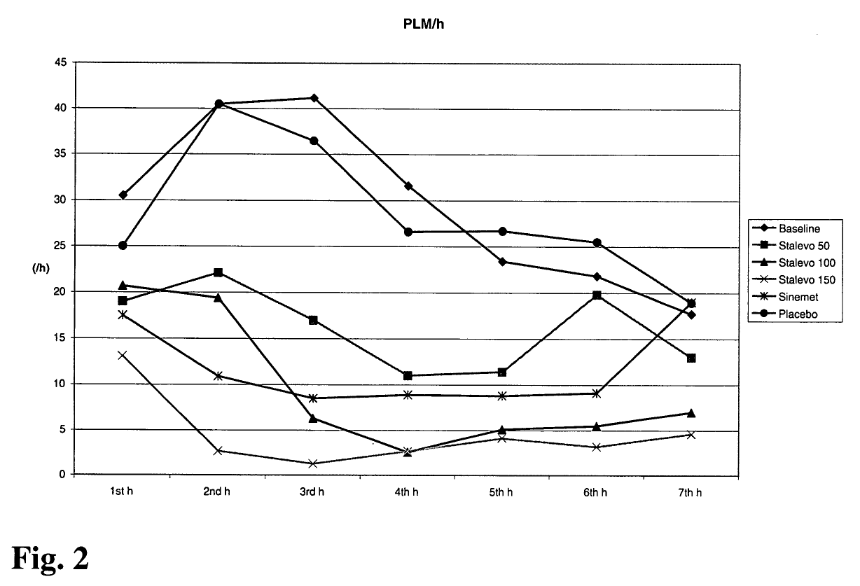

The applicants have discovered that when Stalevo®, a fixed combination of entacapone, a COMT inhibitor, levodopa and carbidopa, a DDC inhibitor, is given to patients with idiopathic RLS, the PLM index of said patients is reduced significantly also during the second half of the night. This is surprising, especially in view of the fact that the kinetics of immediate release levodopa alone or in combination with entacapone does not suggest such a long term action for levodopa therapy especially with such low doses. The effect of Stalevo® containing 100 and 150 mg of levodopa lasted up to 6 and 7 hours, respectively, and both were more effective than standard levodopa/DDCI (levodopa/DDC inhibitor) during the 4 last hours of the night (FIG. 2). The effect of Stalevo® containing 50 mg of levodopa was similar to standard levodopa/DDCI containing 100 mg of levodopa during this period (FIG. 2). The applicants were also surprised to observe that a single dose of Stalevo® containing 150 mg of levodopa was able to control, better than placebo, subjective symptoms of RLS for the week between the doses (Table 1).

Thus, COMT inhibitors, such as entacapone, and their pharmacologically acceptable esters or salts, can be used for the treatment of restless legs syndrome in combination with a dopamine precursor and a DDC inhibitor.

COMT inhibitors of the invention include, without limitation, entacapone and tolcapone, and their analogs and pharmaceutically acceptable esters or salts. Entacapone ((E)-N,N-diethyl-2-cyano-3-(3,4-dihydroxy-5-nitrophenyl)acrylamide) is described in U.S. Pat. No. 5,446,194 and U.S. Pat. No. 5,135,950, which are incorporated herein by reference. Stalevo®, the fixed combination of entacapone, levodopa and carbidopa, is disclosed in EP 1189608 B and Comtess®, the stand alone formulation of entacapone, is disclosed in EP 1112065 B, which are incorporated herein by reference. A dosage form comprising levodopa and carbidopa and entacapone and consisting of an immediate release and a controlled release component is disclosed in US 2004/0166159 A1. Tolcapone (3,4-dihydroxy-4′-methyl-5-nitrobenzophenone) is described in EP 237 929 B.

DDC inhibitors include, without limitation, carbidopa and benserazide. Levodopa and carbidopa are commercially available both as immediate release and slow release (depot) combination tablets sold in Europe under, for instance, the following trademarks: Nacom®, Sinemet® and Sinemet® Plus. Levodopa and benserazide are commercially available both as immediate release and slow release (depot) combination tablets, for instance, under the trademark Madopar®.

Dopamine precursors include, without limitation levodopa and its prodrugs, such as melevodopa, the methyl ester of levodopa, which is disclosed in EP 252290 B.

The precise amount of the drugs to be administered to a patient according to the present invention is dependent on numerous factors known to one skilled in the art, such as the compounds to be administered, the general condition of the patient, the severity of the condition to be treated and the desired duration of use. For example, for Stalevo® and for combinations of entacapone (Comtess®/Comtan®) and levodopa/carbidopa, the usual daily dosage of levodopa will be from 50 mg to 300 mg but can be from 200 mg to 600 mg, divided into 1 to 4, preferably into 1 to 2 individual doses. However, based on the results obtained with the International Restless Legs Study Group (IRLSSG) rating scale, where single dose of Stalevo® containing 150 mg of levodopa was able to control the symptoms of RLS for a week and statistically significantly better than placebo, it is also possible to administer the drugs on-demand, i.e. when needed or required by the emerged symptoms, or once-a-week.

The catechol-O-methyl transferase inhibitor, peripheral decarboxylase inhibitor and dopamine precursor may, in one embodiment, be combined in a single dosage form.

In another embodiment, the agents may be in separate dosage forms. For example, the catechol-O-methyl transferase inhibitor may be in one dosage form and the peripheral decarboxylase inhibitor and dopamine precursor may be combined in a second dosage form. The catechol-O-methyl transferase inhibitor may be taken sequentially or simultaneously with the dopamine precursor and the DDC inhibitor.

Another embodiment includes a kit containing a catechol-O-methyl transferase inhibitor, peripheral decarboxylase inhibitor and dopamine precursor. The kit may contain, for example, the catechol-O-methyl transferase inhibitor in one dosage form and the peripheral decarboxylase inhibitor and dopamine precursor combined in a second dosage form. The kit may contain separate compartments to accommodate separate dosage forms. A patient may access the kit, for example, to take periodic doses of the active agents for the treatment of restless legs syndrome.

It is also possible to use a COMT inhibitor together with an immediate release or slow release combination of levodopa and carbidopa or levodopa and benserazide. In one embodiment of the invention, the daily dose of levodopa is divided into two individual doses, whereby the first dose is an immediate release dosage form.

In another embodiment of the invention, the daily dose of levodopa is divided into two individual doses, whereby the second dose is a slow release dosage form. For example, patients having a severe condition may take a tablet of Comtess® together with an immediate release Sinemet® 1-5 hours before bedtime and another tablet of Comtess® together with a slow release Sinemet® less than 1 hour before bedtime.

In still another embodiment of the invention, the drugs are administered in a single dosage form comprising an immediate release and a slow release component. For example, both components may comprise the peripheral decarboxylase inhibitor and dopamine precursor, whereas the catechol-O-methyl transferase inhibitor may be contained only within one of the components or within both the immediate release and the slow release components.

In another embodiment the drugs are administered on demand. The RLS symptoms may emerge unexpectedly once or twice a week for example when already in bed, during a flight, in a theatre or in a meeting. Dopamine precursor/peripheral decarboxylase inhibitor given in combination with entacapone has fast onset and long duration of action providing symptom relief within the first hour and lasting until morning or until the end of the flight, meeting or theatre.

In one embodiment of the invention, the peripheral decarboxylase inhibitor and dopamine precursor are administered in a ratio from 1:1 to 1:40, such as from 1:4 to 1:10. For example, benserazide and levodopa may be administered in a ratio of 1:4 and carbidopa and levodopa may be administered in a ratio of from 1:4 to 1:10, for instance in a ratio from 1:2 to 1:1.

For the purposes of this invention the term “treatment” means treatment in order to remedy or alleviate the symptoms of the disorder or the condition, and treatment in order to prevent the development or the exacerbation of the symptoms.

For the purposes of this invention the term “on-demand” means treatment taken when the RLS symptoms have unexpectedly emerged and disturb the ongoing activity such as trying to fall asleep, participating a meeting, travelling in an airplane or sitting in a theatre.

The term “immediate release component” used herein encompasses a component in a pharmaceutical formulation that would allow essentially similar release of levodopa from said formulation as immediate release Sinemet®.

The term “slow release component” used herein encompasses a component in a pharmaceutical formulation that would allow essentially similar release of levodopa from said formulation as the slow release formulation Sinemet® Depot.

The invention will be further clarified by the following examples, which are intended to be purely exemplary of the invention, and should not be construed as limiting.

### EXAMPLE 1

In this study the three strengths of the triple combination Stalevo® (i.e. levodopa:carbidopa:entacapone is 50 mg:12.5 mg:200 mg and 100 mg:25 mg:200 mg and 150 mg:37.5 mg:200 mg) were compared against placebo. Standard immediate release Sinemet® 100 (levodopa 100 mg+carbidopa 25 mg) was used as a positive control. At each treatment session, RLS patients received a single dose of treatment and were then monitored overnight for periodic limb movements by polysomnography (PSG). In clinical trials PSG has been a standard methodology for “objective” measurement of PLM in RLS patients.

The primary variable was periodic limb movements/h/total sleep time (PLM Index). The PLM Index has been one of the most common primary variables in short-term studies to document acute drug administration effects. Therefore it is regarded to be a validated, reliable and highly indicative variable to study the effects of Stalevo® in the study. To study the duration of the drug effect, the PLM Index was studied during the first and the second half of the total time in bed (TIB). Comparisons amongst all treatments were performed.

Study outline

| Design: | A randomized, double-blind, placebo-controlled, cross-over, | |
|---|---|---|
| | single-dose, dose-finding and proof-of-concept study with | |
| | STALEVO ® in RLS | |
| Inclusion | Patients with idiopathic RLS according to IRLSSG criteria, | |
| criteria: | who also had at least moderate PLMs (PLM/h in TST or | |
| | TIB >5/h) and were without current dopaminergic | |
| | treatment for RLS | |
| Methods: | Seven sleep laboratory nights with polysomnography (PSG) | |
| | measurement for each subject including two baseline nights | |
| | without any treatment and five nights with randomized study | |
| | treatments (placebo, Stalevo ® 50, | |
| | Stalevo ® 100, Stalevo ® 150 and Sinemet ®). A 4-8 day | |
| | wash-out between the nights. The study | |
| | treatment was given 30 min before bedtime. | |
| Efficacy | I° PLM index (PLMs/h of total sleep time) | |
| Variables: | II° | PLM/h time in bed total and during the first and second |
| | | half of the night. Other PSG variables, safety, subjective |
| | | sleep quality |
| Sample | 28 | |
| size: | | |

TST = total sleep time

Results

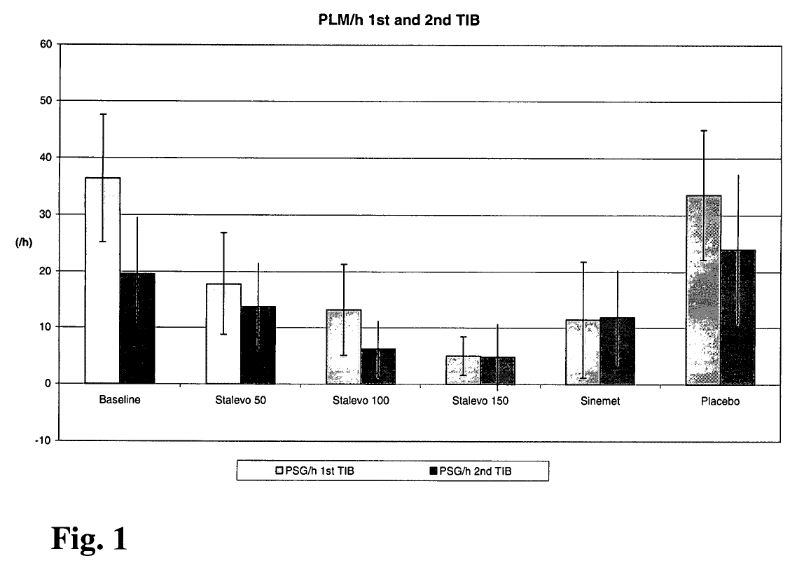

All three doses of Stalevo® were superior to placebo for all PLM variables studied. There was also a significant dose response between the three Stalevo® strengths. It is particularly noteworthy that Stalevo® 100 showed a prolonged activity on PLM in the latter part of the night, compared with standard levodopa 100 mg+DDC inhibitor (FIG. 1).

### EXAMPLE 2

There was a difference in terms of the findings of the IRLSSG rating scale. Stalevo® containing 150 mg of levodopa was more effective than placebo, mean scores 9.3 (Stalevo® 150) and 10.9 (placebo). This scale is generally used in long-term studies as it measures overall RLS symptoms during the previous week. In the present study, the scale was used to assess RLS symptoms between the treatment periods. Summary information on the results from the IRLSSG rating scale is provided in Table 1.

TABLE 1
IRLSSG Rating Scale
Descriptive statistics

| Variable | Baseline | Stalevo ® 50 | Stalevo ® 100 | Stalevo ® 150 | Sinemet ® | Placebo | Statistics |
|---|---|---|---|---|---|---|---|
| Total score | N | 28 | 28 | 28 | 27 | 28 | 28 |
| | Mean | 11 | 10.4 | 11 | 9.3 | 10.3 | 10.9 |
| | Std Dev | 3.2 | 2.8 | 2.7 | 3.3 | 3.8 | 3.3 |
| | Minimum | 6 | 3 | 7 | 1 | 2 | 7 |
| | Median | 11.5 | 11 | 11 | 10 | 10.5 | 10 |
| | Maximum | 18 | 16 | 16 | 14 | 18 | 20 |

Estimates

| Label | Estimate | Std Err | t Value | Pr > \|t\| | Lower | Upper |
|---|---|---|---|---|---|---|
| Stalevo ® 50 vs. Stalevo ® 100 | −0.6152 | 0.5733 | −1.07 | 0.2857 | −1.7522 | 0.5217 |
| Stalevo ® 50 vs. Stalevo ® 150 | 1.0684 | 0.5796 | 1.84 | 0.0682 | −0.08120 | 2.2180 |
| Stalevo ® 50 vs. Sinemet ® | 0.07036 | 0.5740 | 0.12 | 0.9027 | −1.0681 | 1.2089 |
| Stalevo ® 50 vs. Placebo | −0.4587 | 0.5756 | −0.80 | 0.4273 | −1.6002 | 0.6828 |
| Stalevo ® 100 vs. Stalevo ® 150 | 1.6836 | 0.5793 | 2.91 | 0.0045 | 0.5346 | 2.8326 |
| Stalevo ® 100 vs. Sinemet ® | 0.6856 | 0.5737 | 1.20 | 0.2348 | −0.4521 | 1.8233 |
| Stalevo ® 100 vs. Placebo | 0.1566 | 0.5748 | 0.27 | 0.7859 | −0.9834 | 1.2965 |
| Stalevo ® 150 vs. Sinemet ® | −0.9980 | 0.5798 | −1.72 | 0.0882 | −2.1479 | 0.1519 |
| Stalevo ® 150 vs. Placebo | −1.5271 | 0.5814 | −2.63 | 0.0099 | −2.6801 | −0.3740 |
| Sinemet ® vs. Placebo | −0.5290 | 0.5740 | −0.92 | 0.3589 | −1.6675 | 0.6094 |

We claim:

1. A method for the treatment of restless legs syndrome in a mammal, comprising administering to the mammal in need of the treatment an effective amount of a catechol-O-methyl transferase inhibitor, a peripheral decarboxylase inhibitor and dopamine precursor.

2. The method according to claim 1, wherein the restless legs syndrome is idiopathic.

3. The method according to claim 1, wherein the catechol-O-methyl transferase inhibitor is entacapone.

4. The method according to claim 1, wherein the decarboxylase inhibitor is carbidopa.

5. The method according to claim 1, wherein the dopamine precursor is levodopa.

6. The method according to claim 5, wherein the daily dose of levodopa is from 50 mg to 300 mg.

7. The method according to claim 4, wherein carbidopa and levodopa are administered in a ratio from 1:4 to 1:10.

8. The method according to claim 5, wherein the daily dose of levodopa is divided into two individual doses.

9. The method according to claim 8, wherein the first dose of levodopa is an immediate release dosage form.

10. The method according to claim 8, wherein the second dose of levodopa is a slow release dosage form.

11. The method according to claim 1, wherein the three active ingredients are combined in a single dosage form.

12. The method according to claim 1, wherein the active ingredients are administered once-a-week.

13. The method according to claim 1, wherein the active ingredients are administered on demand.
